(19) United States
(12) Patent Application Publication
(10) Pub. No.: US 2012/0283535 A1
(43) Pub. Date: Nov. 8, 2012

(54) METHOD AND SYSTEM FOR PULSE MEASUREMENT

(76) Inventor: Israel Sarussi, Yad Binyamin (IL)

(21) Appl. No.: 13/512,601

(22) PCT Filed: Nov. 30, 2010
(86) PCT No.: PCT/IB10/55504

§ 371 Date: Jul. 26, 2012

Related U.S. Application Data
(60) Provisional application No. 61/264,863, filed on Nov. 30, 2009.

(51) Int. Cl. A61B 5/024; A61B 5/1455; A61B 6/00

(52) U.S. Cl. 600/324; 600/479

(57) ABSTRACT

A method and system for determining a person's heart pulse rate in noisy environments is provided. The method of determining a person's heart pulse rate includes radiating first and second wavelengths of light towards a tissue, measuring and storing a first and second set of parameter values from the signals reflected back from the first and second wavelengths respectively. The first set of parameter values represents a first signal corresponding to a combination of the heart pulse rate and extraneous noise and the second set of parameter values represents a second signal mainly comprising extraneous noise. The heart pulse rate is obtained by deducting the second set of parameter values from the first set of parameter values.

### FIELD OF THE INVENTION

The present invention relates to method and system for the measurement of heart pulse rate or pulse oximetry using reflective pulse oximetry in noisy environments.

### BACKGROUND OF THE INVENTION

The acceptable method in today's market of measuring the pulse during physical activity, such as jogging, running or cycling, for example, is by attaching an electro-cardiograph (ECG) belt around a person's chest. Generally, the ECG belt measures the heart pulse rate and sends a wireless signal to a watch on the person's wrist. This ECG belt is extremely uncomfortable and tends to interfere with the physical activity. Furthermore, the belt is impractical for more strenuous sporting activities and for use in water sports and thus its use is fairly limited.

A further problem associated with the measurement of signals such as the pulse rate during movement is the negative effect of ‘noise’ or extraneous signals which prevent accurate measurements.

Thus, it would be advantageous to be able to measure one's pulse rate without the need for wearing an ECG belt around the chest. It would be further advantageous to measure an optical signal, SPO2 or any other measurement in a noisy environment.

### SUMMARY OF THE INVENTION

The present invention describes embodiments of a method and system for accurately determining the heart pulse rate in situations where the signal being measured is likely to be affected by ‘noise’ and other disturbances.

A method and system for determining a person's heart pulse rate in noisy environments is provided. There is therefore provided, in accordance with an embodiment of the present invention a method of determining a person's heart pulse rate includes radiating first and second wavelengths of light towards a tissue, measuring and storing a first and second set of parameter values from the signals reflected back from the first and second wavelengths respectively. The first set of parameter values represents a first signal corresponding to a combination of the heart pulse rate and extraneous noise and the second set of parameter values represents a second signal mainly comprising extraneous noise. The heart pulse rate is obtained by deducting the second set of parameter values from the first set of parameter values.

Furthermore, in accordance with an embodiment of the present invention, the method further includes the step of: for a sampling taken over a specific time period, comparing the value of the sample for the first signal from the first set of parameter values, with the corresponding value of the second signal from the sample from the second set of parameter values taken over the specific time period; and if there is a difference between the first and second sampling values, altering the first and second sampling values by applying weighting coefficients to each of the first and second sampling values.

Furthermore, in accordance with an embodiment of the present invention, the method further includes the step of applying a Fast Transversal Recursive Least Squares (FTRLS) filter comprising forward and backward prediction filters to each of the first and second sampling values.

Furthermore, in accordance with an embodiment of the present invention, two light sources may be used, each having a different wavelength.

Additionally, there is also provided, in accordance with an embodiment of the present invention, an optical accelerometer which includes first and second light sources for radiating first and second wavelengths of light, respectively towards a tissue, a sensor for receiving a first signal and a second signal reflected back from the tissue from the first and second light sources respectively and a filter. The first signal includes an AC signal representing the heart pulse rate together with a DC signal and the second signal includes a DC signal representing movement plus a relatively minor AC signal. The filter acts to separate the first received AC signal from the second received DC signal, thereby to determine the movement.

Furthermore, in accordance with an embodiment of the present invention, the filter is an adaptive filter configured to applying weighting coefficients to the AC and DC signals. The filter includes a Fast Transversal Recursive Least Squares (FTRLS) filter including forward prediction and backward prediction filters.

Additionally, there is also provided, in accordance with an embodiment of the present invention a non-invasive device for the measurement of heart pulse rate and pulse oximetry. The device includes first and second light sources for radiating first and second wavelengths of light, respectively towards a tissue; a sensor for receiving light reflected back from the tissue from the first and second light sources, the received light from the first light source represents a first signal corresponding to a combination of the heart pulse rate and extraneous noise and the second set of parameter values represents a second signal corresponding to the extraneous noise only; and a filter configured for separating the signal representing the heart pulse rate from signal representing a combination of the heart pulse rate and noise, thereby to obtain the heart pulse rate.

Furthermore, in accordance with an embodiment of the present invention, the filter includes an adaptive filter for comparing a first value of the first signal from the first set of parameter values, with the corresponding second value of the second signal from the second set of parameter values taken over a specific time period; and if there is a difference between the first and second values, altering the first and second values by applying weighting coefficients to each of the first and second sampling values.

Furthermore, in accordance with an embodiment of the present invention, the filter further includes a Fast Transversal Recursive Least Squares (FTRLS) filter. The FTRLS filter includes a forward prediction filter and a backward prediction filter.

Furthermore, in accordance with an embodiment of the present invention, a combination of two lights sources having low AC values are used in order to separate the ‘noise’ from the ‘signal+noise’, each light source having a different wavelength.

### BRIEF DESCRIPTION OF THE DRAWINGS

The present invention will be understood and appreciated more fully from the following detailed description taken in conjunction with the appended drawings in which:

FIG. 1 is a schematic block diagram illustration of a method for pulse measurement in accordance with an embodiment of the invention;

FIG. 2A is a schematic graphical illustration representing the measurement of the signal plus noise, in accordance with an embodiment of the invention;

FIG. 2B is a schematic graphical illustration representing the measurement of the noise only, in accordance with an embodiment of the invention;

FIG. 2C is a schematic graphical illustration representing the desired signal, in accordance with an embodiment of the invention

FIG. 3 is a schematic block diagram illustration of an adaptive filter used with the method of FIG. 1, in accordance with an embodiment of the invention;

FIG. 4 is a flow chart illustration of the method in accordance with an embodiment of the invention; and

FIG. 5 is a schematic block diagram of the components of a non-invasive device for the measurement of heart pulse rate and pulse oximetry, utilizing the method of FIG. 1.

It will be appreciated that for simplicity and clarity of illustration, elements shown in the drawings have not necessarily been drawn accurately or to scale. For example, the dimensions of some of the elements may be exaggerated relative to other elements for clarity or several physical components included in one functional block or element. Further, where considered appropriate, reference numerals may be repeated among the drawings to indicate corresponding or analogous elements. Moreover, some of the blocks depicted in the drawings may be combined into a single function.

### DESCRIPTION OF THE INVENTION

In the following description, various aspects of the present invention will be described. For purposes of explanation, specific configurations and details are set forth in order to provide a thorough understanding of the present invention. However, it will also be apparent to one skilled in the art that the present invention may be practiced without the specific details presented herein. Furthermore, well known features may be omitted or simplified in order not to obscure the present invention.

The present invention relates to method and system for the measurement of heart pulse rate or pulse oximetry using reflective light, such as reflective pulse oximetry.

This standard optical method of transmitted pulse oximetry transmits a beam of light from one side of the finger to the other side. On the opposite side of the finger, a detector measures the amount of light that has not been absorbed by the blood in the finger. The detected beam represents the heart beat. This transmitted method cannot be used on the wrist since the amount of energy required to send the beam and receive results are extremely large and not practical.

The present invention uses reflective pulse oximetry, similar to the method described in U.S. Pat. No. 7,171,251 assigned to the present applicants. Different optical methods may also be used with the present invention.

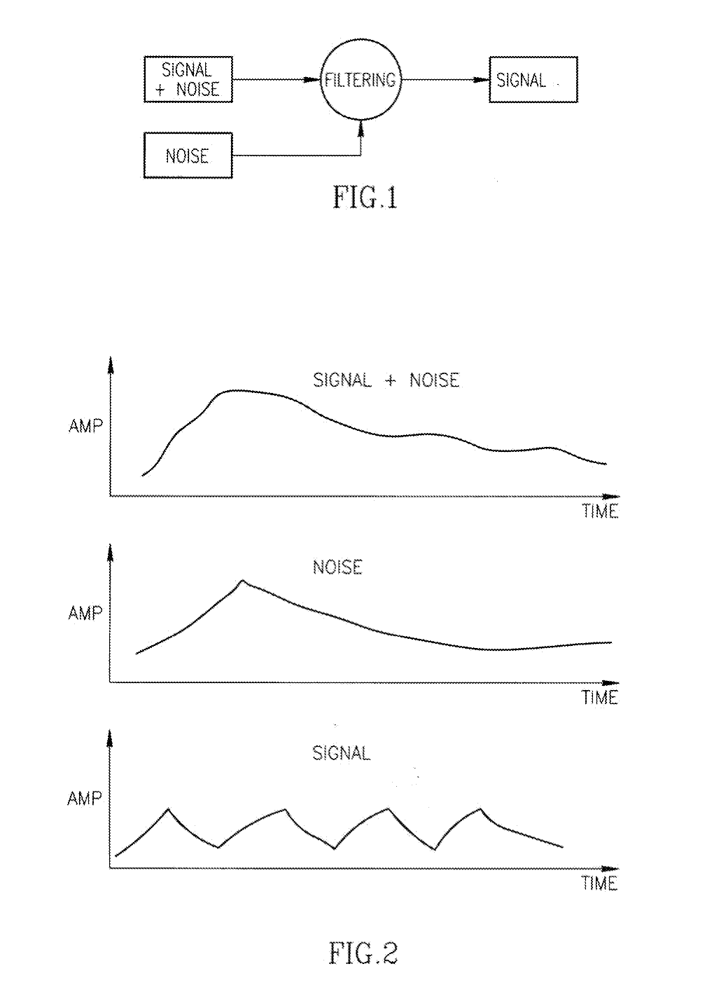

Reference is now made to FIGS. 1-4, which illustrate the method for the measurement of heart pulse rate from a wrist, in accordance with an embodiment of the invention. For exemplary purposes only, the present invention refers to the pulse rate measurements from a person's wrist. However, it will be appreciated that the description of the present invention is may apply to any part of the body.

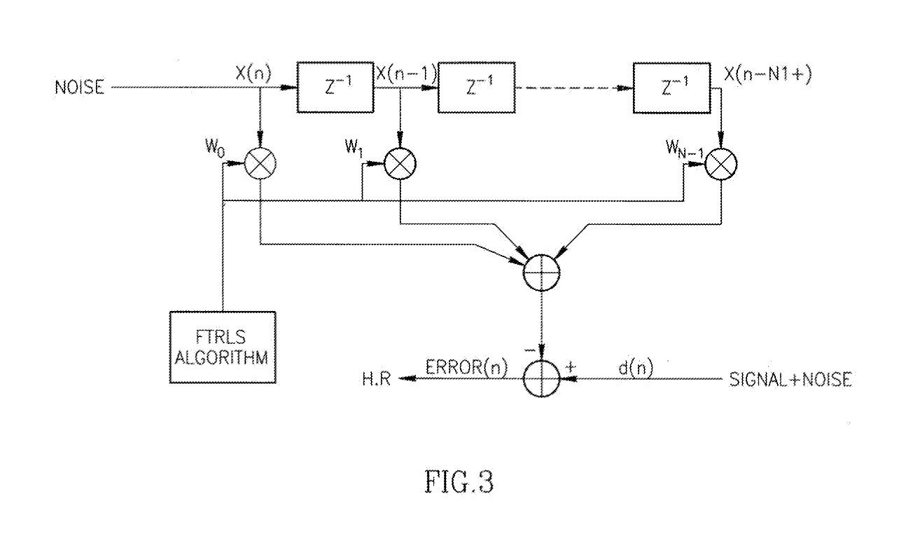

FIG. 1 is a schematic block diagram illustration of a method for pulse measurement in accordance with an embodiment of the invention. FIG. 2A is a schematic graphical illustration representing the measurement of the signal plus noise together. FIG. 2B is a schematic graphical illustration representing the measurement of the noise only. FIG. 2C is a schematic graphical illustration representing the desired signal, in accordance with an embodiment of the invention. FIG. 3 is a schematic block diagram illustration of an adaptive filter used with the method of the present invention.

In order to measure the reflected light from a wrist, a special sensor with an extremely high level of dynamic sensitivity is required.

The signal measured from the wrist contains information concerning the heart beat and additional information concerning the movements of the muscles and nerves and other disturbances (noise). It is important to filter out these disturbances especially during physical activity and other movements.

The inventors have realized that the signal may be separated from the surrounding noise by only measuring the noise and deducting this measurement from a second measurement which includes the signal and noise, as schematically shown in FIG. 1.

The method may be described as follows:
The sensor takes at least two measurements for every sampling. The first measurement measures the whole signal including all of the noise and the entire signal. The second measurement is a measurement of the noise. The second measurement may also contain a small portion of the heart rate, but is effectively almost zero and thus for mathematical purposes it may be ignored. The measurements may be taken in any order.
The noise measurement is then deducted from the first measurement and the result is the “clean” heart beat (pulse measurement).

It is important to note that the subtraction is not a vector subtraction but the subtracting of the forms or shapes representing the frequencies of the two measurements. Each measurement comprises an array (or set) of parameter values which may be represented by a graphical wave shape, similar to a sinusoidal graph, for example.

In an exemplary embodiment of the invention a suitable number of measurements (between 32-500 times, for example) of the two signals are taken. Each measuring cycle may produce a different result. If all of the sampled points in one cycle (4 to 6 seconds, for example) are added together, a wave representing the movement is produced. A clean signal would expect to be illustrated by a shape comprising a six wave sinusoidal form, for example.

Embedded in this wave are the heart beats, which are invisible because of the great ratio between them. Thus, when the signal wave (including the signal and the movement) and the wave of only the movements (the noise referred to above) are added together, almost a full congruence is obtained. That is a correlation of almost 1 is achieved.

The expected signal representing pulse measurement is in the approximately sinusoidal shape illustrated in FIG. 2C. However, any disturbance (‘noise’) during measurement will result in a distorted shape shown in FIG. 2A, which is an exemplary illustration of the “signal+noise” shape. The measurement of ‘noise’ only may be carried out and an exemplary illustration of the “noise” shape is shown in FIG. 2B.

Both of the waves, that is “signal+noise” and ‘noise only’ are put into an adaptive filter (FIG. 3) that sets to zero all fully overlapping waves (all of the waves that correlate close to 1). Thus, the remaining waves represent the heart beats that are left over from the first wave (first measurement).

As shown in FIG. 3, the adaptive filter uses a FTRLS (Fast Transversal Recursive Least Squares) algorithm which utilizes a combination of lights having low AC values to separate the ‘noise’ from the ‘signal+noise’. For example, blue light having a wavelength of 450-475 nm may be used to represent ‘noise’ in combination with green light (representing signal+noise) having a wavelength of 495-570 nm. Alternatively, a combination of red (having a wavelength of 620-750 nm) and infra-red (having a wavelength of 700-1400 nm) may be used. It will be appreciated that any combination of two different frequencies with a small, wavelength between them may be used to separate noise from the signal.

For each sample, a comparison of the ‘noise’ (X(n)) with the ‘signal+noise’ (d(n)) is made to estimate the pulse (Error (n)). If the signal is not clear, that is, there is a difference between the two samples, indicating that extraneous noise is present, the FTRLS algorithm within the adaptive filter processes the signals and applies weighting coefficients (W0, W1, Wn−1) to each of the signals received.

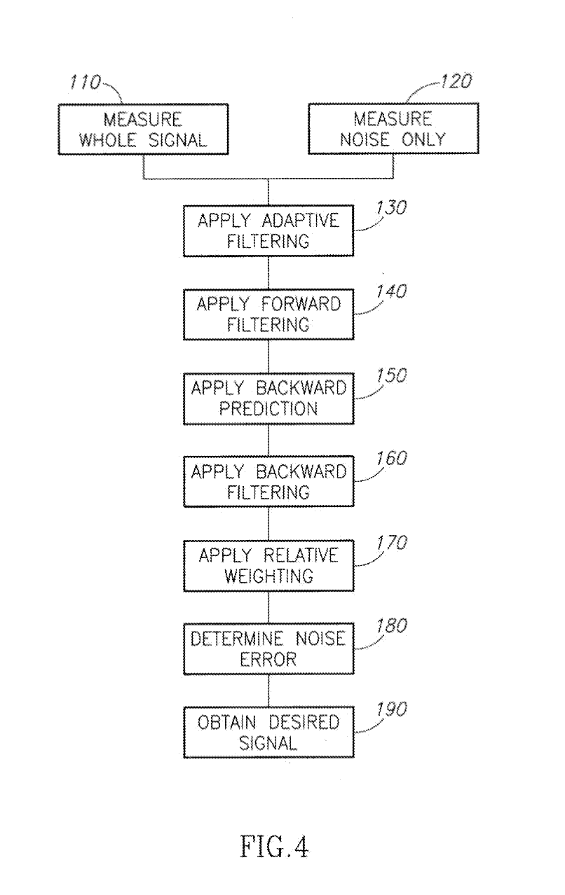

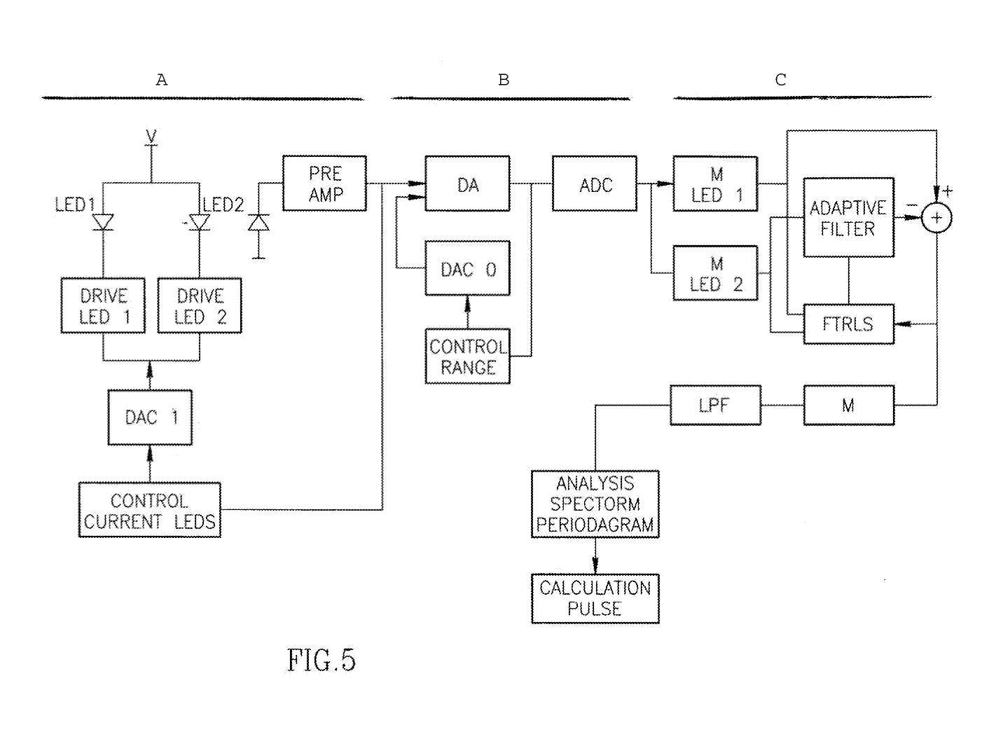

Reference is now made to FIG. 4, which is a flow chart illustration of the method in accordance with an embodiment of the invention.

In operations 110 and 120, two measurements are taken for every sampling. The first measurement 110 measures the whole signal including the all of the noise and the entire signal. The second measurement 120 measures the noise only. The measurements may be taken in any order.

In operation 130, adaptive filtering, such as Kalman filtering utilizing a Fast Transversal Recursive Least Squares (FTRLS) algorithm, for example, is applied, as described hereinabove with reference to FIG. 3. Kalman filtering may be used since it applicable for measurements over time and can compensate for noise distortion by using weighted averages to produce values closer to the true values of the measurements.

The FTRLS algorithm may use the following computations, for example, to obtain the desired clean signal:

For each input sample (X):

X(j)=X(j−1) (1)

Where: X is an array of the current and last N samples.

In operation 130, the adaptive (Kalman) filtering may update the samples as follows:

X(1)=x(n); (2)

g=x(n); (3)

g=g+A(j)+X(j); (4)

m(1)=−g*alpha; (5)

m(1)=C(j−1)+m(1)*A(j); (6)

h=gamma1−m(1)*g; (7)

where: g is a priori forward prediction error;
A is a forward prediction filter;
m=Kalman gain of order N+1;
h=gamma1 of order N+1;
C is the Kalman gain; and
gamma1=1/gamma;

In operation 140, forward filtering is applied as follows:

e=g*gamma; (8)

A(j)=A(j)+e*C(j−1); (9)

Alpha=alpha−m(1)*m(1)/h; (10)

where: e is a posteriori forward prediction error;
alpha is the inverse of least squares sum of forward errors.

In operation 150, further modification is made using a backward prediction filter, as follows:

rpf=0; (11)

rpf=rpf+B(j)*X(j); (12)

C(j)=m(j)−m(N+1)*B(j); (13)

s=1/(h+m(N+1)*rpf; (14)

r=rpf*s; (15)

where: rpf is a priori backward prediction error;
B is a backward prediction filter;
s=cosine of oblique angle;
r is a posteriori backward prediction error

In operation 160, backward filtering is applied as follows:

e=g*gamma; (16)

B(j)=B(j)+r*C(j); (17)

beta=beta+r*rpf; (18)

gamma=beta*alpha; (19)

gamma1=1/gamma; (20)

where: beta=least square sum of backward prediction errors.

In operation 170, relative weighting is applied as follows:

XX(j)=XX(j−1); (21)

XX(1)=x(n); (22)

epsp=d(n); (23)

epsp=epsp+W(j)*XX(j); (24)

eps=epsp*gamma; (25)

W(j)=W(j)+eps*C(j); (26)

Error(n)=eps. (27)

Where: XX is equivalent to, but not reinitialized;
epsp is a priori output error;
W is the desired impulse response;
eps is a posteriori output error;
d is the current desired response sample (IR signal

The result of the above computations lead to a calculation of the noise error (operation 180). By deducting the noise error form the total “signal+noise” sample, a clean signal representing the pulse may be obtained (step 190).

The present invention may be applied in many applications where it is necessary to measure ‘noise’ to obtain accurate readings. For example, present invention may be used in an optical accelerometer to exclusively measure ‘noise’. The optical accelerometer may have the following characteristics:
The response to movement will be exactly the same response to the measured signal;
The measured signal contains a minimum pulse signal.

Basically, the above is achieved by using a signal that is very similar to an optic signal. Light having wave lengths which have different absorption rates in the blood are preferably used. When light is radiated towards tissue, for example, some of the light is absorbed by the blood. The remainder of the light is reflected back to a sensor. The reflected signal includes the heart beat, which is effectively the pulse rate.

The received signal contains a high DC, representing the movement, plus an AC signal representing the minimal heart beats. The DC signal relates to a constant movement, while the heart beat signal varies, Thus, since we wish to calculate the movement, and not the heart beat, lights having lower wavelengths should be used. Lower wavelengths produce a larger DC component relative to the AC component.

It will be appreciated that light sources having wave lengths, which are different from than the usual wave length used for optic measuring may be used. In an exemplary embodiment of the invention, blue and green light, or any other combination of two lights may be used.

In an exemplary embodiment of the invention a suitable number of measurements (between 32-500 times, for example) for 4-30 seconds or more, for example of the two signals are taken. Each measuring cycle may produce a different result.

As described hereinabove, the receive signals may be processed using an adaptive filter, such as a Kalman Filter together with the Fast Transversal Recursive Least Squares (FTRLS) filter to obtain an accurate measurement of the movement.

Reference is now made to FIG. 5, which is a schematic block diagram of a non-invasive device, such as a wrist watch for measuring the heart pulse rate using reflective pulse oximetry. The device may comprise three sections. Section “A” illustrates the components for controlling the light intensity. The light intensity increases as the signal becomes smaller. Section “B” illustrates the components required for separating the AC signal from the DC signal, similar to that described in U.S. Pat. No. 7,171,251 assigned to the present applicants. Section “C” illustrates the components required for separation of the signal from the noise. The components of section “C:” include the adaptive filter using FTRLS algorithm, for example, described hereinabove with reference.

The foregoing description of the embodiments of the invention has been presented for the purposes of illustration and description. It is not intended to be exhaustive or to limit the invention to the precise form disclosed. It should be appreciated by persons skilled in the art that many modifications, variations, substitutions, changes, and equivalents are possible in light of the above teaching. It is, therefore, to be understood that the appended claims are intended to cover all such modifications and changes as fall within the true spirit of the invention.

1. A method for determining a person's heart pulse rate, comprising:
radiating first and second wavelengths of light towards a tissue;
measuring and storing a first and second set of parameter values from the signals reflected back from said first and second wavelengths respectively, wherein said first set of parameter values represents a first signal corresponding to a combination of the heart pulse rate and extraneous noise and said second set of parameter values represents a second signal mainly comprising extraneous noise;
deducting the second set of parameter values from the first set of parameter values, thereby to obtain the heart pulse rate.

2. The method of claim 1, further comprising the steps of:
for a sampling taken over a specific time period, comparing the value of the sample for the first signal from said first set of parameter values, with the corresponding value of the second signal from said sample from said second set of parameter values taken over said specific time period; and
if there is a difference between the first and second sampling values, altering said first and second sampling values by applying weighting coefficients to each of said first and second sampling values.

3. The method of claim 2, further comprising the step of:
applying a Fast Transversal Recursive Least Squares (FTRLS) filter comprising forward and backward prediction filters to each of the first and second sampling values.

4. The method of claim 1, wherein said first light has a first wavelength and said second light has a second wavelength wherein the second wavelength is different from said first wavelength.

5. An optical accelerometer comprising:
first and second light sources for radiating first and second wavelengths of light, respectively towards a tissue;
a sensor for receiving a first signal and a second signal reflected back from the tissue from said first and second light sources respectively, said first signal comprising an AC signal representing the heart pulse rate together with a DC signal and said second signal comprising a DC signal representing movement plus a relatively minor AC signal; and
a filter configured for separating the first signal from the second signal, thereby to determine the movement.

6. The optical accelerometer of claim 5, wherein said first light source has a first wavelength and said second light source has a second wavelength wherein the second wavelength is different from said first wavelength.

7. The optical accelerometer of claim 5, wherein said filter is an adaptive filter configured to applying weighting coefficients to said first and second signals.

8. The optical accelerometer of claim 7, wherein said filter comprises a Fast Transversal Recursive Least Squares (FTRLS) filter, said FTRLS filter comprising a forward prediction filter and a backward prediction filter.

9. A non-invasive device for the measurement of heart pulse rate and pulse oximetry, comprising:
first and second light sources for radiating first and second wavelengths of light, respectively towards a tissue;
a sensor for receiving light reflected back from the tissue from said first and second light sources, said received light from said first light source represents a first signal corresponding to a combination of the heart pulse rate and extraneous noise and said second set of parameter values represents a second signal corresponding to the extraneous noise only; and
a filter configured for separating the signal representing the heart pulse rate from signal representing a combination of the heart pulse rate and noise, thereby to obtain the heart pulse rate.

10. The device of claim 9, wherein said filter comprises:
an adaptive filter for comparing a first value of the first signal from said first set of parameter values, with the corresponding second value of the second signal from said second set of parameter values taken over a specific time period; and
if there is a difference between the first and second values, altering said first and second values by applying weighting coefficients to each of said first and second sampling values.

11. The device of claim 10, wherein said filter further comprises a Fast Transversal Recursive Least Squares (FTRLS) filter, said FTRLS filter comprising a forward prediction filter and a backward prediction filter.

12. The device of claim 9, wherein said first light has a first wavelength and said second light has a second wavelength wherein the second wavelength is different from said first wavelength.
